(19) United States
(12) Patent Application Publication
(10) Pub. No.: US 2001/0050992 A1
(43) Pub. Date: Dec. 13, 2001

(54) Two-piece wireless electromechanical corpometer/stethoscope

(76) Inventor: Timothy Patrick Carman, La Jolla, CA (US)

Correspondence Address: TIMOTHY CARMAN, 6365 CASTEJON DRIVE, LA JOLLA, CA, 92037, US

(21) Appl. No.: 09/876,495

(22) Filed: Jun. 7, 2001

Related U.S. Application Data
(60) Provisional application No. 60/210,782, filed on Jun. 12, 2000.

(51) Int. Cl. A61B 7/04

(52) U.S. Cl. 381/67

(57) ABSTRACT

A means of combining a handheld electromechanical transducer, amplification and radio wave transmitter unit with a paired headset containing a like-frequency radio wave receiver, amplification unit and earphones for the purpose of improving on the ability of health practitioners to auscultate biological sounds in the course of clinical medical practice.

### CROSS-REFERENCE TO RELATED APPLICATIONS

A Provisional Patent Application for this instrument/invention has been filed by the inventor, Timothy P. Carman with a filing date of Jun. 12, 2000 (date received by the PTO), and this application claims the benefit of those documents so received. As of this writing I have not received a corresponding serial number from the PTO for that submitted PPA, however it's application Ser. No. is 60/210,782.

### STATEMENT REGARDING FEDERALLY SPONSORED RESEARCH OR DEVELOPMENT

“Not Applicable”

### REFERENCE TO A MICROFICHE APPENDIX

“Not Applicable”

### BACKGROUND OF THE INVENTION

The field of use of this invention is in Clinical Medical Practice. Specifically, this invention serves to improve upon deficiencies present (both appreciated and unappreciated) in the current instrument in the field whose use it is intended to supplant; the stethoscope.

A limiting factor long present in the current/prior art is in the sound quality afforded by the nature of the design of all traditional stethoscopes currently in use. Specifically, a mechanical diaphragm monitors bodily sounds and transmits them mechanically via sound waves by way of a hollow tube. For adequate appreciation of the quality of sounds auscultated, one required two conditions, namely, a quiet environment and the absence of any objects hitting against the sound propagating tube. Constant attempts at improving the current stethoscope as designed (in an effort to overcome the first of these two conditions, but not the second), have been offered. Yet, there has been little improvement in the sound quality (in terms of volume and tone, and the users' ability to control those two parameters) available today as present in most stethoscope designs over it's originally invented form, namely, a single piece unit incorporating the air-conducting tube.

Another cumbersome property of the current /prior art of the stethoscope design is its bulky size. It is well appreciated that the stethoscope fits poorly in the labcoat pocket, hence, its ever-present draping around most practitioners' necks, which in itself can be a nuisance.

Accordingly, several objects and advantages of my invention over the traditional air-conduction stethoscope can be appreciated. First, the two piece design allows for greater freedom of movement for the health practitioner while examining the patient. The traditional stethoscope involves listening via the mechanical conduction of sound through a rubber tube that has a fixed length. The practitioner must always be able to position his head in close proximity to the diaphragm portion of the traditional stethoscope, often leaning over the patient in an uncomfortable position.

Another object and advantage is the improvement of sound quality in terms of both volume and tone characteristics over the traditional sound conduction stethoscope. Because there is no physical tube, the Two Piece Corpometer/Stethoscope will not suffer from the interfering noises that occur when objects or personnel in close proximity to the examiner hit the rubber tubing of a traditional air-conduction stethoscope. Also, the headset portion of my invention contains separate volume and tone controls to adjust sound levels to suit the needs of the practitioner as well as compensate for a noisy environment, as in the case of a busy Emergency Room.

Further objects and advantages of my invention will become apparent from a consideration of the drawings and ensuing description.

### BRIEF SUMMARY OF THE INVENTION

The “Two Piece Corpometer/Stethoscope” or “Two Piece Carmascope” as it may be called, has the following objects and advantages, and solves problems inherent with previous stethoscope designs as follows:

1) Compact and efficient design. It is designed in two pieces; a hand held portion and a headset portion. The handheld portion fits comfortably in the hand during use, and given its compact size, fits easily in the normal trouser pocket, let alone the large pockets of the traditional white lab coat. The headset portion is very lightweight (as compared with a traditional stethoscope) and when worn during use fits comfortably in both ears. When not in use, it can be worn unobtrusively around the neck.

2) Improved sound quality. The handheld portion has a diaphragm similar to that of a traditional stethoscope, however it also contains electronic circuitry that converts the mechanical energy of the auscultated sound waves picked up by the diaphragm into radio waves, and then sends them (via transmitter which is also part of the circuit in the handheld portion) to the headset. The headset contains a receiver that receives those sound waves for listening by the user. Also present on the headset portion are separate volume and tone controls, which allow the user to overcome any interfering environmental noise (for example, the large amount of noise present in an emergency room) as well as adjust tonal qualities to accentuate features of the sounds to which he/she is listening. As well, because there is no intervening tube needed to conduct sound, there is no chance of extraneous sounds (produced by objects or people present hitting against that tube) interfering with the sounds to which the examiner is listening.

3) Improved ease of use. An unexpected advantage in this two-piece design is the elimination of the distance restriction inadvertently present with a device whose listening portion (diaphragm) is connected [via the sound tube] to its ear portion, i.e., the stethoscope in its current form. Specifically, the examiner's head must be in close proximity to the patient when using the traditional stethoscope. This can be especially difficult in the emergency room setting where a large number of individuals surround the patient at one time. The proposed design of this two-piece invention allows for a much more flexible approach to the patient in real-life clinical situations.

### BRIEF DESCRIPTION OF THE SEVERAL VIEWS OF THE DRAWING

FIGURES

FIG. 1a shows handheld portion of Two Piece Corpometer/Stethoscope, top view.

FIG. 1b shows handheld portion of Two Piece Corpometer/Stethoscope, bottom view.

FIG. 2 shows handheld portion of Two Piece Corpometer/Stethoscope, exploded bottom view.

FIG. 3 shows headset portion of Two Piece Corpometer/Stethoscope

FIG. 4a and 4b show simplified schematic representations of the handheld and headset portions of the Two Piece Corpometer/Stethoscope, respectively.

### REFERENCE NUMERALS IN DRAWINGS

10 Lightweight Metal Housing, Handheld Portion

11 Finger Recesses In Handheld Portion

12 Activation, or “On-OFF” Button On Handheld Portion

13 Plastic Diaphragm of Handheld Portion

14 Metal Retaining Bracket of Handheld Portion

15 Retaining Screws, Handheld Portion

16 Housing containing Electromechanical Transducer and Amplifier Circuit of Handheld Portion

17 Radio wave Transmitter of Handheld Portion

18 Headset Assembly Housing

19 Headset Earpiece, Left

20 Headset Earpiece, Right

21 Headset Volume Control

22 Headset Tone Control

23 Headset Amplifier Circuit/Radio Wave Receiver Module

24 Electromechanical Transducer of Handheld Portion, Schematic Representation

25 Amplifier Circuit of Handheld Portion, Schematic Representation

26 Battery of Handheld Portion, Schematic Representation

27 Radio wave Transmitter of Handheld Portion, Schematic Representation

28 Radio wave Receiver of Headset Portion, Schematic Representation

29 Amplifier Circuit of Headset Portion, Schematic Representation

30 Volume Control of Headset Portion, Schematic Representation

31 Tone Control of Headset Portion, Schematic Representation

32 Earphone of Headset Portion, Schematic Representation

33 Battery of Headset Portion, Schematic Representation

### DETAILED DESCRIPTION OF THE INVENTION

The “Two Piece Corpometer/Stethoscope” or “Two Piece Carmascope” as it may be called, can be described as being comprised of electrical and electromechanical components the nature and construction of which are well appreciated by those persons well skilled in the field(s) of electronic and mechanical engineering. The uniqueness and utility, along with unexpected advantages of the proposed invention lies in its creative COMBINATION of those elements to produce an instrument which substantially improves (see BACKGROUND OF THE INVENTION) upon prior art, specifically, the air conduction stethoscope.

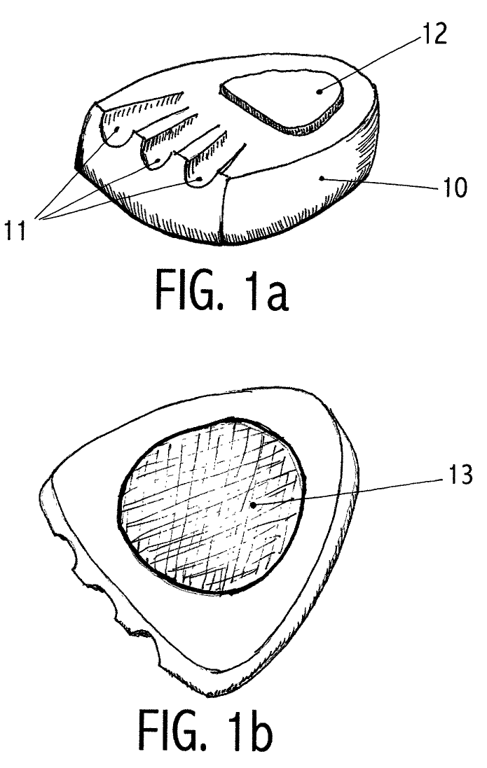

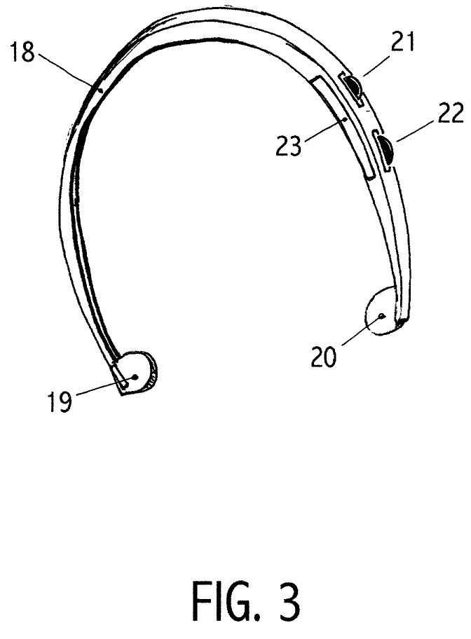

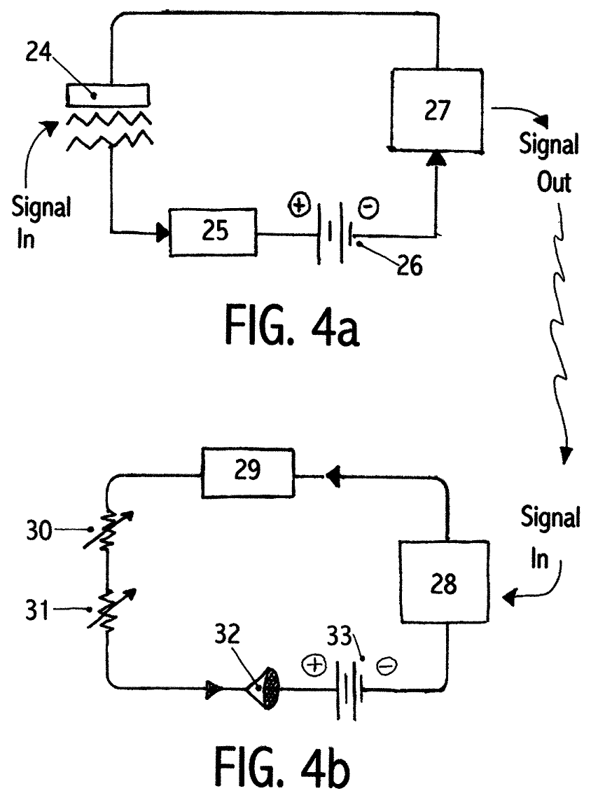

The first unit of the two piece embodiment is the hand held portion (FIGS. 1a,1b and 2). It is comprised of a lightweight metal housing 10 that contains a thin plastic diaphragm 13, such as that used in a traditional stethoscope, an electromechanical transducing portion 16, 24, which converts mechanical sound vibrations to electrical impulses and is similar to a diaphragm type microphone. Also present in the housing are an amplification circuit 16, 25 that receives the signal from the transducer 24. The final circuit in the housing is a simple transmitter 17, 27, which transmits the signal to its like-frequency receiver 23, 28, in the second portion of the Two Piece Corpometer, the headset (FIG. 3).

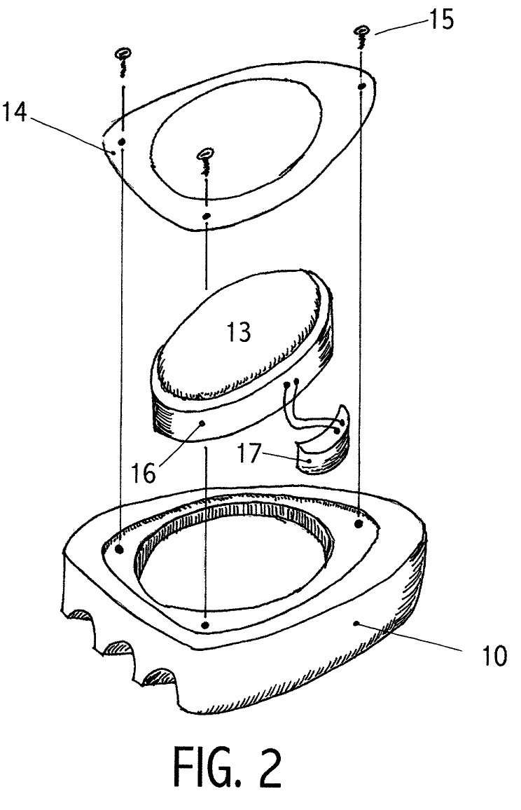

The headset (FIG. 3) is made of a lightweight flexible high impact plastic material 18 not unlike similar designs used in home or entertainment audio configurations. It contains a matched radio receiver 23, 28 that receives the signal generated from the hand held portion's transmitter 17, 27 (FIGS. 1a, 1b and 2). The signal is then transferred to an amplifier circuit 23, 29. The headset's amplifier circuit 23, 29 contains two adjustment knobs or wheels, which are potentiometers that separately control volume 21, 30, and tone 22, 31, sound qualities. The signal then arrives at the two earpieces 19, 20, 32, where the listener appreciates the sounds auscultated by the hand held portion (FIGS. 1a, 1b and 2).

I Claim:

1. A means of combining a handheld diaphragm based electromechanical transducer, amplification circuit, unique radio wave transmitter and a headset earphone radio wave receiver together as a paired apparatus, in such a manner as to improve upon the sound quality and ease or flexibility of use in the clinical examination and auscultation of patient generated biological sounds over that of the current and prior art, the traditional air-conduction stethoscope.

2. A two piece listening or auscultation medical device comprising:
a) a handheld portion which contains an electromechanical transducer, amplification circuit, and radio wave transmitter, and
b) a headset portion containing a paired radio wave receiver, amplification circuit and earphones for sound reproduction of auscultated biological sounds.
